(19) United States
(12) Patent Application Publication
(10) Pub. No.: US 2026/0347067 A1
(43) Pub. Date: Nov. 5, 2026

(54) CLEANING AGENT CARTRIDGE

(71) Applicant: Saban Ventures Pty Limited, Macquarie Park, NSW (AU)

(72) Inventors: Amir Hossein KAYVANPOUR, Carlingford, NSW (AU); David Anthony PIDCOCK, Dulwich Hill, NSW (AU); Rigor Del Mundo ASPA, Sutherland, NSW (AU); Makomo TSUBONO, Dulwich Hill, NSW (AU); Phuong Hy Ton TRAN, Strathfield, NSW (AU); Joanne Aguirre MERAM, Marsfield, NSW (AU)

(21) Appl. No.: 19/115,486

(22) PCT Filed: Dec. 8, 2022
(86) PCT No.: PCT/AU2022/051469

§ 371 Date: Mar. 26, 2025

(30) Foreign Application Priority Data
Sep. 26, 2022 (AU) 2022902786

(51) Int. Cl. B65D 47/32; B65D 47/06

(52) U.S. Cl. CPC B65D 47/32; B65D 47/06; B65D 2217/00

(57) ABSTRACT

Presented herein are cleaning agent cartridges for storing and FIG. 8B transporting a cleaning agent, techniques for mixing the cleaning agent with a fluid within a cleaning agent cartridge, and techniques for dispensing the resultant fluidic cleaning composition from the cleaning agent cartridge.

### BACKGROUND

### Cross-Reference to Related Applications

This application claims priority to Australian Patent Application No. 2022902786, entitled “Dry Powder Cartridge,” filed Sep. 26, 2022, the content of which is hereby incorporated by reference herein.

### Field of the Invention

The present invention generally relates to cartridges for storing and transporting a cleaning agent, mixing the cleaning agent with a fluid, and dispensing a resultant fluidic cleaning composition.

### Related Art

There are a number of devices/systems that include internal channels/lumens that need periodic or regular cleaning. For example, there are different types of medical devices (medical instruments) that can be used to perform diagnostic and/or surgical procedures. An endoscope is one such medical device that can be used to visually inspect hollow organs or body cavities. Specially designed endoscopes are used for different examinations, such as bronchoscopy, cystoscopy, gastroscopy, and proctoscopy. Endoscopes, as well as other available diagnostic and/or surgical medical devices are re-useable across multiple patients and include one or more interior lumens that must be cleaned between uses.

### SUMMARY

In one aspect, a cleaning agent cartridge is provided. The cleaning agent cartridge comprises: a tank configured to retain a dry powder; a closure assembly configured to be attached to the tank to form a closed volume; at least one first port disposed in the closure assembly configured for ingress of a fluid into the tank; a riser tube extending from the at least one first port into the tank; and one or more second ports disposed in the closure assembly configured for ingress of a fluid into, and egress of fluid from, the tank.

In another aspect, a method is provided. The method comprises: fluidically coupling at least one first port and one or more second ports of a cartridge to a cleaning device, wherein the cartridge forms a closed container including at least one powder; introducing fluid into the cartridge via the at least one first port; introducing fluid into the cartridge via the one or more second ports, wherein the fluid and powder form a fluidic cleaning composition; and dispensing the fluidic cleaning composition from the cartridge via at least one of the one or more second ports.

In another aspect, a method is provided. The method comprises: fluidically coupling a cartridge to a cleaning device, wherein the cartridge forms a closed container including at least one powder; introducing fluid from a first hydration locus within the cartridge; introducing fluid from a second hydration locus within the cartridge, wherein the fluid and the at least one powder form a fluidic cleaning composition within the cartridge; and dispensing the fluidic cleaning composition from the cartridge.

In another aspect, a cartridge for storage of a dry powder, for hydration of the dry powder to provide a slurry, and for dispensing said slurry; the cartridge comprising: a closure assembly and a tank closed by the closure assembly, the closure assembly comprising at least a first port, operable to permit passage of a fluid into the tank or passage of a gas out from the tank, and one or more second ports, operable to permit passage of a fluid into the tank and passage of a slurry from the tank, the tank adapted to retain the dry powder, wherein a conduit extends from the first port within the tank to define a first hydration locus and the one or more second ports define a second hydration locus.

### BRIEF DESCRIPTION OF THE DRAWING

Embodiments of the present invention are described herein in conjunction with the accompanying drawings, in which:

FIG. 1 is a schematic diagram illustrating an endoscope with interior lumens that can be cleaned in association with a cleaning agent cartridge, in accordance with certain embodiments presented herein.

FIG. 2A is a flowchart of an example method for cleaning an interior lumen of a medical device using a fluidic cleaning composition dispensed from a cleaning agent cartridge, in accordance with certain embodiments presented herein.

FIG. 2B is a schematic diagram illustrating a first stage/phase of a process for cleaning a lumen with a fluidic cleaning composition.

FIG. 2C is a schematic diagram illustrating a second stage/phase of a process for cleaning a lumen with a fluidic cleaning composition.

FIG. 3 is a schematic diagram illustrating use of a cleaning agent cartridge with a lumen cleaning device, in accordance with certain embodiments presented herein.

FIG. 4 is a cross-sectional diagram of a cleaning agent cartridge, in accordance with certain embodiments of presented herein.

FIG. 5 is a cross-sectional diagram of the cleaning agent cartridge of FIG. 4, shown with a travel cap, in accordance with certain embodiments of presented herein.

FIG. 6 is a cross-sectional diagram of another cleaning agent cartridge, in accordance with certain embodiments of presented herein.

FIGS. 7A, 7B, 7C, and 7D are a series of schematic diagrams illustrating operation of a cleaning agent cartridge with a lumen cleaning device for mixing and dispensing a hydrated solid powder (fluidic cleaning composition) , in accordance with certain embodiments presented herein.

FIGS. 8A, 8B, 8C, 8D, 8E, and 8F are a series of diagrams illustrating operation of another cleaning agent cartridge for mixing and dispensing a hydrated solid powder (fluidic cleaning composition), in accordance with certain embodiments presented herein.

FIG. 9 is block diagram illustrating an example computing device configured to operate as a control system for hydration of a powder and dispensing of a resultant fluidic cleaning composition, in accordance with certain embodiments presented herein.

FIG. 10 is a flowchart illustrating an example method, in accordance with certain embodiments presented herein.

FIG. 11 is a flowchart illustrating another example method, in accordance with certain embodiments presented herein.

### DETAILED DESCRIPTION

Presented herein are cleaning agent cartridges for storing and transporting cleaning agents (e.g., dry powders, fluids etc.), techniques for mixing the cleaning agent with a fluid within the cleaning agent cartridge, and techniques for dispensing resultant fluidic cleaning compositions from the cleaning agent cartridge. A fluidic cleaning composition can be used to, for example, clean the internal channel/lumen of a system/device. It is to be appreciated that the cleaning agent cartridges and associated techniques can be used with any of a number of different systems/device, such as medical devices with internal channels/lumens, food/beverage service lines, etc. However, merely for ease of illustration, the techniques will primarily be described with reference to the cleaning of endoscope lumens.

An endoscope is an elongate tubular medical device that can be rigid or flexible and which incorporates an optical or video system and light source. Typically, an endoscope is configured so that one end can be inserted into the body of a patient via a surgical incision or via one of the natural openings of the body. Internal structures near the inserted end of the endoscope can thus be viewed by an external observer.

As well as being used for investigation, endoscopes are also used to carry out diagnostic and surgical procedures. For example, endoscopy is used worldwide for the screening, diagnosis, and treatment of gastrointestinal (GI) diseases, among others, and enables early detection and treatment. Endoscopic procedures are increasingly popular as they are minimally invasive in nature and provide a better patient outcome (through reduced healing time and exposure to infection) enabling hospitals and clinics to achieve higher patient turnover.

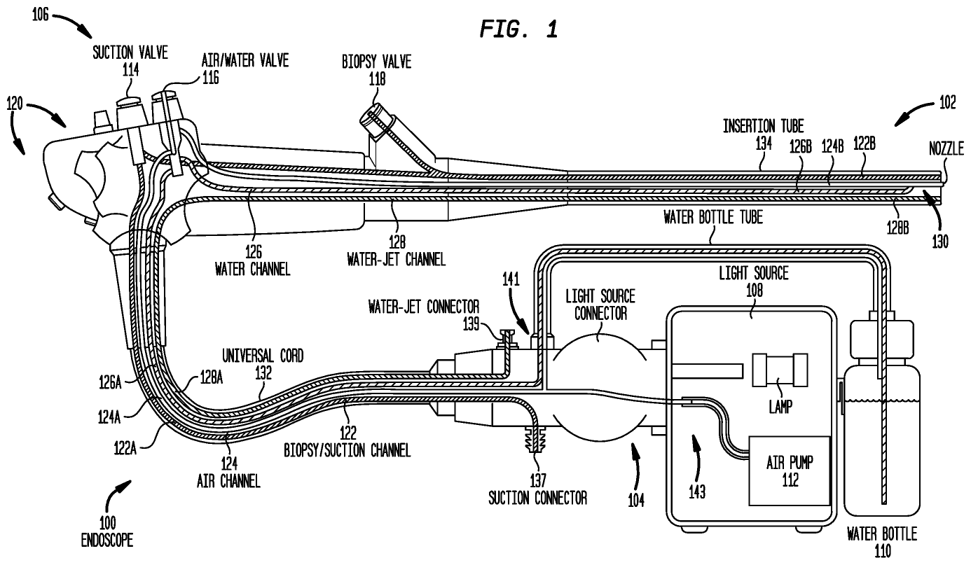

FIG. 1 is a schematic diagram of an example endoscope 100 with which aspects of the techniques presented herein can be implemented. As shown, endoscope 100, similar to most endoscopes, has a long tube-like structure with a distal end/tip 102 at one end for insertion into a patient and an opposing proximal or connector end 104, with a control handle 106 located between the two ends (e.g., generally at the center of the length between connector end 104 and distal end 102). The connector end 104 includes a plurality of connectors that enable the endoscope to be attached to, for example, a light source 108, water source 110, a suction source (not shown in FIG. 1), and a pressurized air source 112. For example, shown in FIG. 1A, is a suction port/connector 137, a water-jet (auxiliary) port/connector 139, a water port/connector 141, and an air-port/connector 143. The control handle 106 is held by the operator during the procedure to control the endoscope 100 via valves, which include in this example a suction valve 114, an air/water valve 116, and a biopsy valve 118, and control wheels 120.

As shown in FIG. 1, endoscope 100 includes internal channels used either for delivering air and/or water, providing suction or allowing access for forceps and other medical equipment required during the procedure. As such, the distal tip 102 contains the camera lens (not shown in FIG. 1), and the exits for the lighting, air, and water, as well as exits for suction and forceps. Some of the internal channels run from one end of the endoscope 100 to the other, while others run via valve sockets at the control handle. Some channels bifurcate while and others join from two into one.

More specifically, shown in FIG. 1 is a biopsy/suction channel 122, an air channel 124, a water channel 126, and a water-jet channel 128. The biopsy/suction channel 122 includes two sections, referred to as proximal section 122A and distal section 122B that are connected via the suction valve 114. The air channel 124 also includes two sections, referred to as proximal section 124A and distal section 124B that are connected via the air/water valve 116. Similarly, the water channel 126 also includes two sections, referred to as proximal section 126A and distal section 126B that are connected via the air/water valve 116. The distal section 126B of the water channel joins to the distal section 124B of the air channel at a location 130 within the distal end 102. The water-jet channel 128 extends directly from the connector end 104 to the distal end 102 (via the control handle 106) but is similarly referred to as having a proximal section 128A and distal section 128B. The proximal sections 122A, 124A, 126A, and 128A of the channels are sometimes referred to as being located within a universal cord section (cord) 132 of the endoscope 100, while the distal sections 122B, 124B, 126B, and 128B of the channels are sometimes referred to as being located within an insertion tube 134 of the endoscope. More generally, as used herein, the proximal sections 122A, 124A, 126A, and 128A are the portions of the channels located between the connector end 104 and a valve (e.g., valve 114 or 116) at the control handle 106 and/or a mid-point of the control handle 106, as applicable. The distal sections 122B, 124B, 126B, and 128B are the portions of the channels located between the valve (e.g., valve 114 or 116) at the control handle 106 and/or a mid-point of the control handle 106, and the distal end 102 of the endoscope 102.

The high cost of endoscopes means they must be re-used. As a result, because of the need to avoid cross infection from one patient to the next, each endoscope must be thoroughly cleaned and disinfected or sterilized after each use. This involves the cleaning of not only the outer of the endoscope 100, but also cleaning and disinfecting the internal channels/lumens (e.g., lumens 122, 124, 126, and 128 of FIG. 1).

Endoscopes used for colonoscopic procedures are typically between 2.5 and 4 meters long and have one or more lumen channels of diameter of no more than a few millimeters. Ensuring that such long narrow channels are properly cleaned and disinfected between patients presents a considerable challenge. The challenge of cleaning is also made more difficult by the fact that there is not just one configuration/type of endoscope. Indeed, there are a variety of endoscopic devices, each suited to a particular insertion application, such as colonoscopes inserted into the colon, bronchoscopes inserted into the airways, gastroscopes for investigation of the stomach, etc. Gastroscopes, for instance, are smaller in diameter than colonoscopes; bronchoscopes are smaller again and shorter in length while duodenoscopes have a different tip design to access the bile duct.

A variety of options are available to mechanically remove biological residues from the lumen which is the first stage in the cleaning and disinfection process. By far the most common procedure for cleaning the lumens utilizes small brushes mounted on long, thin, flexible lines. Brushing is the mandated technique of cleaning the lumen in some countries. These brushes are fed into the lumens while the endoscope is submerged in warm water and a cleaning solution. The brushes are then pushed/pulled through the length of the lumens in an effort to scrub off the soil/bio burden. Manual back and forth scrubbing is typically required. Water and cleaning solutions are then flushed down the lumens. These flush-brush processes are repeated three times or until the endoscope reprocessing technician is satisfied that the lumen is clean. At the end of this cleaning process air is pumped down the lumens to dry them. A flexible pull-through device having wiping blades can also be used to physically remove material. A liquid flow through the lumen at limited pressure can also be used.

In general, however, only the larger suction/biopsy lumens (e.g., 122 in FIG. 1) can be cleaned by brushing or pull throughs. Air/water channels (e.g., channels 124 and 126) may be too small for brushes so these lumens are usually only flushed with water and cleaning solution.

After mechanical cleaning, a chemical clean can be carried out to remove the remaining biological contaminants. Because endoscopes are sensitive and expensive medical instruments, the biological residues cannot be treated at high temperatures or with strong chemicals. For this reason, the mechanical cleaning needs to be as thorough as possible. In many cases, the current mechanical cleaning methodologies fail to fully remove biofilm from lumens, particularly where cleaning relies on liquid flow alone. Regardless of how good the conventional cleaning processes are, it is almost inevitable that a small microbial load will remain in the channel.

There has been significant research to show that the method of cleaning with brushes, even when performed as prescribed, does not completely remove biofilm in endoscope lumens. As well as lacking in efficacy, the current manual brushing procedures suffer from other drawbacks. The large number of different endoscope manufacturers and models results in many minor variations of the manual cleaning procedure. This has led to confusion and ultimately poor compliance in cleaning processes. The current system of brushing is also hazardous in that the chemicals that are currently used to clean endoscopes can adversely affect the reprocessing staff.

The current system of manual brushing is also labor intensive, leading to increased cost. Thus, the current approaches to cleaning and disinfecting the lumens in medical cleaning apparatus are still inadequate and residual microorganisms are now recognized as a significant threat to patients and staff exposed to these devices. For example, there is evidence of bacterial transmission between patients from inadequate cleaning and disinfection of internal structures of endoscopes which in turn has led to patients acquiring mortal infections. Between 2010 and 2015 more than 41 hospitals worldwide, most in the U.S., reported bacterial infections linked to the scopes, affecting 300 to 350 patients (http://www.modernhealthcare.com/article/20167415/NEWS/167419935). It would be expected that a reduction in the bioburden in various medical devices would produce a concomitant overall reduction in infection rates and mortality.

In addition, if endoscopes are not properly cleaned and dried, biofilm can build up on the lumen wall. Biofilms start to form when a free-floating microorganism attaches itself to a surface and surrounds itself with a protective polysaccharide layer. The microorganism then multiplies, or begins to form aggregates with other microorganisms, increasing the extent of the polysaccharide layer. Multiple sites of attachment can in time join up, forming significant deposits of biofilm. Once bacteria or other microorganisms are incorporated in a biofilm, they become significantly more resistant to chemical and mechanical cleaning than they would be in their free-floating state. The organisms themselves are not inherently more resistant, rather, resistance is conferred by the polysaccharide film and the fact that microorganisms can be deeply embedded in the film and isolated from any chemical interaction. Any residual biofilm remaining after an attempt at cleaning quickly returns to an equilibrium state and further growth of microorganisms within the film continues. Endoscopes lumens are particularly prone to biofilm formation. They are exposed to significant amounts of bioburden, and subsequent cleaning of the long narrow lumens is quite difficult due to inaccessibility and the inability to monitor the cleaning process.

There is considerable pressure in medical facilities to reprocess endoscopes as quickly as possible. Because endoscopes are cleaned by hand, training and attitude of the technician are important in determining the cleanliness of the device. Residual biofilm on instruments can result in a patient acquiring an endoscope acquired infection. Typically, these infections occur as outbreaks and can have fatal consequences for patients.

Although manual cleaning procedures, as described above, have been used for many years, systems and methods have more recently be provided to overcome or ameliorate at least one of the disadvantages of the prior art, or at least to provide a useful alternative. In particular, the use of certain fluidic cleaning compositions propelled through respective lumens of a medical device has been found to be particularly effective at safely removing unwanted matter via physical interaction with the lumens. In certain such techniques, a fluidic cleaning composition in the form of “slurry” is created, apportioned into a suitable amount, and then delivered at a suitable velocity through at least a portion of the lumen. In one form, the apportioned amount of the slurry is sometimes referred herein to as a ‘cleaning slug’ or ‘slug.’

As used herein, the term “fluidic cleaning composition” refers to a fluidic solution that can remove (e.g., physically detach, dissolve, etc.) contaminants. The fluidic cleaning composition can be a saturated or unsaturated solution; and a fluidic cleaning composition in some instances can include solid particles, e.g., where a dry powder cleaning agent is present in a solvent above the respective saturation limit. As used herein, the term “slurry” refers a fluidic solution (e.g., a specific fluidic cleaning composition) that includes solid (e.g., powder) particles suspended in a fluid.

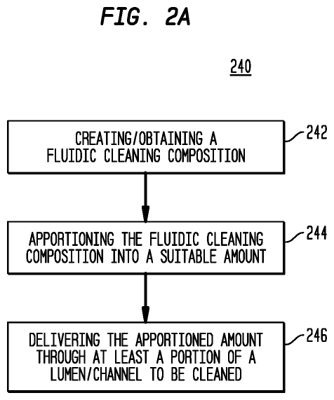

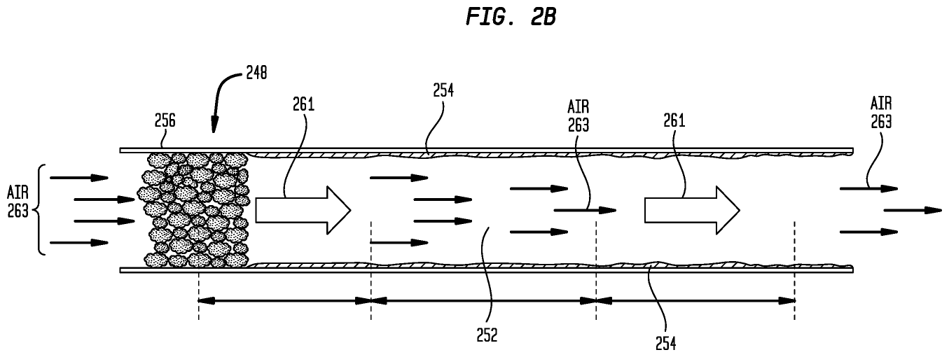

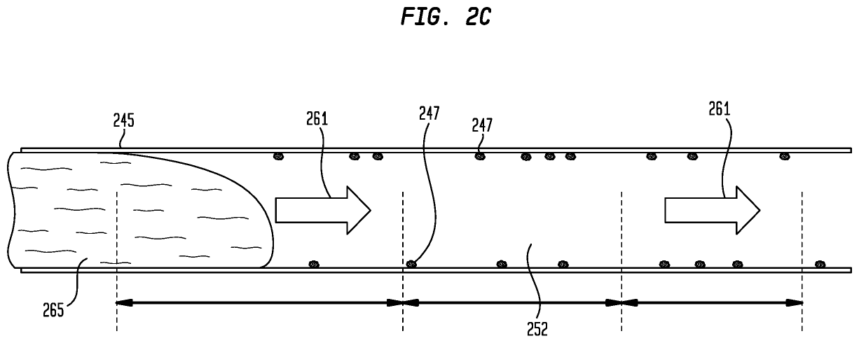

FIG. 2A illustrates an exemplary method 240 of cleaning a lumen of a medical device using a fluidic cleaning composition in the form of a slurry and a cleaning slug. The method 240 of FIG. 2A begins at 242 with the creating, mixing, or otherwise obtaining of the slurry. At 244, the slurry is apportioned into a suitable amount. At 246, the apportioned amount of the slurry (i.e., a cleaning slug) is delivered (e.g., propelled) through at least a portion of a lumen to be cleaned. This process can of course be implemented in any of a variety of ways.

For example, any suitable slurry can be implemented. In one example, the slurry has a liquid component and a powder component. The liquid component of the slurry can facilitate the fluidity of the composition, while the presence of the powder can act to interact with the walls of the target lumen (e.g., channel) to thereby clean the lumen. In accordance with certain examples, the powder component of the slurry is present in amounts greater than the respective saturation limit within the respective liquid, which can facilitate a cleaning interaction between the mixture and the walls of the lumen. In certain embodiments, the slurry comprises a mixture of sodium bicarbonate powder and water, where the sodium-bicarbonate is present in an amount greater than the respective saturation level. For example, in a number of embodiments, sodium-bicarbonate can, at certain stages, be present in an amount greater than 10% of the mixture by mass. It has been determined that a mixture of sodium bicarbonate and water can be particularly effective in the disclosed application. Moreover, these constituent components are readily available. However, it would be appreciated that any suitable slurry can be used in alternative examples.

In some arrangements, the powder in the slurry is present in an amount below the respective saturation of the associated liquid. However, the liquid is delivered to the target lumen prior to the complete dissolution of the powder in the liquid. In this way, the undissolved powder can still interact with the target lumen to be cleaned.

Moreover, it is to be appreciated that the slurry can be created/obtained in any of a variety of ways. For example, in certain embodiments, a powder is obtained from a cartridge or other consumable chamber/container, water is obtained from a tap, and these constituent components are mixed within a holding chamber (or within the consumable chamber itself) proximate (e.g., within days or weeks) to the time of cleaning. This approach can be advantageous insofar as powders such as sodium bicarbonate can be relatively stable and can have a long shelf life and suitable sources of water are readily available. However, in other embodiments, the slurry can be obtained in an already mixed form.

As noted, method 240 involves apportioning the slurry into a suitable amount, sometimes referred to herein as a cleaning slug. As illustrated, the apportioned amounts are subsequently delivered through a lumen to be cleaned. Delivering discrete amounts of the slurry can be advantageous insofar as the discrete amounts can be delivered periodically at suitable velocities, and the periodic application of the composition can help facilitate the cleaning of the lumen while not clogging/blocking the target lumens. Moreover, the discrete nature of the delivered amounts can facilitate the maintenance of a suitable delivery velocity, which can also aid cleaning. For example, if the slurry was delivered continuously (and not in discrete, apportioned amounts), this approach might risk ‘clogging’ or otherwise obstructing the lumen to reduce the velocity at which the slurry flows through the lumen, and can thereby impact cleaning efficacy.

Notably, different amounts of slurry can be differently suitable for the different characteristics of lumens to be cleaned. For example, air/water channels within an endoscope are typically amongst the narrowest lumens and, accordingly, may be more suitably cleaned with relatively smaller amounts of a slurry (whereas using larger amounts of a slurry may result in blocking such a narrow channel). In contrast, the suction/biopsy channels of an endoscope are typically amongst the widest lumens and, accordingly, may be more suitably cleaned with larger amounts of slurry. As such, the amount of slurry apportioned for use in cleaning a given lumen is a function of the geometry of the lumen to be cleaned. It should of course be appreciated that the amount of slurry apportioned can also or alternatively be a function of any of a variety of parameters, including those that relate to the target.

The apportioned amount of the slurry can be determined in any of a variety of ways. For example, in certain embodiments, a valve can be used to draw a target amount of slurry from a reservoir, such as a cleaning agent cartridge described elsewhere herein. In some embodiments, a self-regulating pressurized system is used to draw a suitable amount of slurry from the reservoir.

As noted, method 240 of FIG. 2A further includes delivering the apportioned amount of slurry through at least a portion of the lumen to be cleaned. In general, a carrier fluid (e.g., air, water, etc.) is used to deliver (e.g., propel) the apportioned amount of the slurry through at least a portion of the lumen to be cleaned at a suitable velocity. The apportioned amount of the slurry is delivered in a manner (e.g., suitable size, suitable velocity, etc.) to provide an appropriate physical interaction between the mixture and the walls of the lumen, meaning that the undissolved powder will physically contact or run against the walls of the lumen to remove contaminants (e.g., bioburdens) therefrom. Of course, the apportioned amount of the slurry can be delivered through the lumen in any suitable way to enable cleaning of a lumen.

Notably, method 240 can be iterated any number of times to facilitate the cleaning of the lumen of a medical device. For example, FIG. 2B illustrates the delivery of one cleaning slug 248 (e.g., an apportioned amount of a slurry) through a lumen 252 to remove contaminants from the walls of the lumen, where the general direction of travel of the slug 248 is represented by arrows 261. That is, as shown, the lumen 252 has one or more contaminants 254 (e.g., bioburdens) disposed on the inner surface/walls 256 of the lumen. It is further illustrated that the cleaning slug 248 is delivered through the lumen 252 to physically interact with the walls of the lumen and thereby remove the contaminants 254 therefrom. The cleaning slug 248 can be considered to be entrained within a carrier fluid, which in this example comprises air (represented by arrows 263).

In general, cleaning slugs presented herein, such as cleaning slug 248, can have different forms/arrangements. For example, in certain embodiments, a cleaning slug presented herein can be a relatively singular/unitary mass (e.g., potentially substantially occluding the lumen while traveling therethrough), which is sometimes referred to herein as a “unitary slug.” However, in other embodiments, a cleaning slug can be an “agglomeration” or “cluster” of smaller masses/groups that travel through the lumen as a loose group (e.g., potentially not occluding the lumen while traveling therethrough), sometimes referred to herein as a “cluster slug.” FIG. 2B schematically illustrates an example in which the slugs 248 are cluster slugs.

In certain embodiments, a cleaning slug can transition between different forms during the slug's life cycle. For example, a slug could be apportioned (initially created) as a unitary slug, but then transition to a cluster slug. This transition could occur before entering the lumen (e.g., in a delivery chamber) and/or while traveling through the lumen.

As noted above, FIG. 2B generally illustrates the delivery of a cleaning slug 248 through a lumen 252. In certain examples, FIG. 2B represents a first stage/phase of a cleaning process, while FIG. 2C represents a second stage/phase of the cleaning process. More specifically, after a cleaning slug 248 is delivered through the lumen 252 (as in FIG. 2B), a fluid flow is delivered through the lumen 252 without any slugs. In the example of FIG. 2C, the fluid flow is comprised of water 265, where the general direction of travel is again represented by arrows 261. In certain examples, the fluid flow (e.g., water 265) is configured to remove residuals 247 from the lumen. The residuals 247 can comprise, for example, some remaining portion of the contaminant 254 and/or portions of the slugs 248 that may remain on the walls of the lumens 252 after passage of the slugs (e.g., the slug can break up into different clusters, some of which remain on the walls of the lumen 252). If present, portions of the slug 248 that may remain on the walls of the lumens 252 may aid in the cleaning process as these portions are flushed through the lumen 252 by the fluid flow.

FIGS. 2B and 2C generally illustrate an arrangement in which the second stage (fluid flow) is interspersed between the delivery of cleaning slugs. That is, in the embodiments of FIGS. 2B and 2C, the delivery of each cleaning slug is followed by a fluid-only flow. In certain alternative embodiments, multiple slugs could alternatively be delivered through a lumen either simultaneously or sequentially, without separation (e.g., without a fluid-only flow).

It should be appreciated that any number of cleaning slugs may be delivered through the lumen in different embodiments. In general, the use of a series of discrete/individual cleaning slugs 248, as opposed to a single large flow, can allow the individual cleaning slugs to maintain sufficient kinetic energy to pass through the lumens at a rate that allows the particles with the slugs to advantageously interact with, and remove contaminants from, the lumen walls.

As noted above, a lumen cleaning process, such as described above with reference to FIGS. 2A, 2B, and 2C, can be implemented in a number of different manners with a number of different lumens. For context, one specific example implementation is described with reference to cleaning at least part of the endoscope 100 of FIG. 1A.

More specifically, in one example cleaning process/cycle, one (1) cleaning slug is fired/shot into the water-jet channel 128 via water-jet connector 138, nine (9) cleaning slugs are then fired into the biopsy/suction channel 122 via suction connector 137, one (1) cleaning slug is then fired into the water-jet channel 128 via water-jet connector 138, three (3) cleaning slugs are then fired into the distal section 122B of the biopsy/suction channel 122 via biopsy valve 128, one (1) cleaning slug is then fired into the water-jet channel 128 via water-jet connector 138, and then nine (9) cleaning slugs are fired into the biopsy/suction channel 122 via suction connector 137. The cleaning cycle can further include firing/shooting six (6) cleaning slugs into the air channel 124 via air connector 143 and firing six (6) cleaning slugs into the water channel 126 via water connector 141 (e.g., in parallel). The firing of the cleaning slugs within each target lumen can be followed by a fluid flow, as described above with reference to FIG. 2C. The cleaning slugs and fluid flows can be delivered via one, or possible multiple connectors (e.g., one connector for the air pipe and one connector for the air/water bottle).

In certain examples, approximately 180-200 grams of a slurry could be used to clean a typical flexible GI endoscope. For example, approximately use 80-100 grams can be used to clean a relatively large channel (e.g., suction/biopsy channel 122) with 21 shots in total and an approximately 15 second delay between each shot. For a relatively small channel (e.g., air/water channels), the process can use approximately 60-80 grams with 12 shots in total and an approximately 30 second delay between each shot. For other small channels (e.g., water-jet channel 128), the process can use approximately 10-20 grams with 3 shots in total and an approximately 30 second delay between each shot. Again, each of these channels can also receive a subsequent fluid flow (e.g., after each cleaning slug), as described above with reference to FIG. 2C.

As noted above, cleaning slugs are delivered to a target lumen with a velocity that is suitable/sufficient to remove contaminants from the walls of the target lumen. The velocity of the cleaning slugs can vary, for example, based on the attributes of the target lumen, the attributes of the of the slurry used to form the cleaning slug, etc. In one illustrative example, the slug velocity for a relatively large lumen can be around 1000 mm/second.

In addition, the cleaning slugs can be delivered within specific pressure and fluid flow (air) ranges. In certain examples, the cleaning slugs can be delivered with a pressure up to approximately 26 psi (air, note this is regulated by a PPR as described below), up to approximately 24 psi (water), etc. Example air flow metrics can include approximately 50 SLPM (large channel no load), approximately 11-17 SLPM (large channel during dosing), approximately 7-10 SLPM (large channel during full load), approximately 5-7 SLPM (small channel no load), and approximately 0.1 SLPM (small channel during full load). It is to be appreciated that these ranges and values are merely illustrative. Further details relating to the cleaning of medical device lumens are disclosed in the present Applicant's PCT/AU2022/050568, entitled “Systems and Methods for Cleaning Lumens with Fluidic Compositions” filed on 9 Jun. 2022, the contents of which are hereby incorporated by reference.

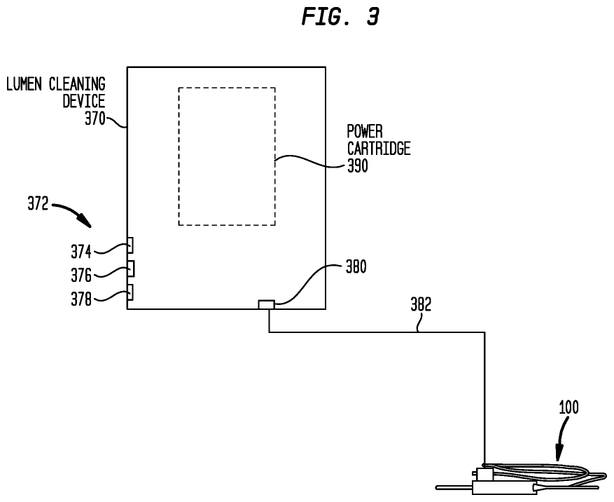

FIG. 3 is a schematic diagram illustrating a lumen cleaning device/apparatus 370 with which embodiments presented herein can be implemented. The lumen cleaning device 370 includes a plurality of connectors 372. The connectors 372 include at least one gas connector 374 for connection of the lumen cleaning device 370 to a gas supply (e.g., compressed dry air supply), at least one water connector 376 for connection of the lumen cleaning device 370 to a water supply (e.g., potable water supply), and a device drain fitting 378.

As shown, the lumen cleaning device 370 also includes an interface/connector 380 for an endoscope adapter hose 382. The endoscope adapter hose 382 connects the lumen cleaning device 370 to one or more lumens of an endoscope, such as endoscope 100.

As noted above, in an example automated lumen cleaning process, such as one implemented by the lumen cleaning device 370, a powder is mixed with a liquid to form a slurry, which is then apportioned and caused to flow through at least one internal channel/lumen of the medial device to remove the bioburden adhered to the walls of the lumen. In the example of FIG. 3, the lumen cleaning device 370 is configured to interface with a cleaning agent cartridge 390 (schematically represented in FIG. 3 by a dashed box) that is configured for storing, transporting, and mixing a dry powder with a liquid (e.g., water) to form a slurry.

In FIG. 3, the cleaning agent cartridge 390 is shown within the lumen cleaning device 370. However, it is to be appreciated that this specific relationship between the cleaning agent cartridge 390 and the lumen cleaning device 370 is merely illustrative. For example, in other embodiments the cleaning agent cartridge 390 can be located partially or fully external to the lumen cleaning device 370 (e.g., interface with external components of the lumen cleaning device 370). Further details of example cleaning agent cartridges for interface within a lumen cleaning device, such as lumen cleaning device 370, are provided below.

For ease of description, aspects of the techniques presented herein will generally be described with reference to a cleaning agent in the form of a powder, and the dispensing of a fluidic cleaning composition in the form of a slurry. However, as described elsewhere herein, the techniques presented herein can be used with other types of cleaning agents and other types of fluidic cleaning compositions.

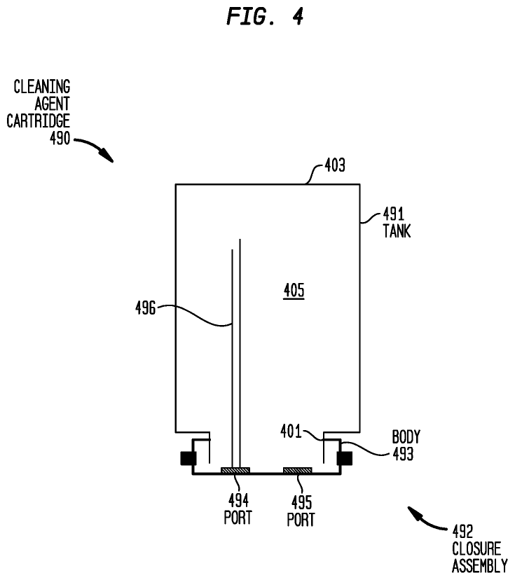

More specifically, FIG. 4 is a schematic diagram illustrating a cleaning agent cartridge 490 comprising a tank portion (tank) 491 and a closure assembly 492 in accordance with various embodiments of the invention. As shown, the tank 491 comprises a first (proximal) end 401, a second (distal) end 403, and defines an interior/internal volume 405. As shown, the closure assembly 492 comprises a body 493 having at least two ports therein, referred to as a first port 494 and a second port 495. The body 493 is configured to be attached to the first end 401 of the tank 491, and the ports 494 and 495 are configured so as to close the internal volume 405 of the cartridge 490 (i.e., the region inside the tank 491) from uncontrolled contact with the atmosphere, water or other contaminants, while being operable to permit material to move in and/or out of the cartridge when required by a user or, for example, at a predetermined time in an automated cleaning cycle.

For ease of illustration, only a single first port 494 and a single second port 495 are shown in FIG. 4. However, it is to be appreciated that multiple first ports and/or multiple second ports can be present. As described further below, the first port(s) and second port(s) can be used to allow ingress and egress of fluids to e.g., further aid in the hydration of the solid powder and/or the dispensation of the slurry.

As described further below, the cleaning agent cartridge 490 is configured to be attached to the cleaning device which can then access the cartridge contents via the ports 494 and 495. The ports 494 and 495 retain the powder in the cartridge 490 until the cartridge contents are dispensed by the cleaning device in a controlled manner. In certain embodiments, the ports 494 and 495 can each comprise a port member configured to, at least initially, retain the powder within the cartridge 490. The port member can be, for example, a pressure activated valve, a frangible membrane impermeable to fluids, etc.

More specifically, in one embodiment, the ports 494 and 495 each have a valve (e.g., a valve with a preferential direction, a bi-directional valve, etc.) which is actuated by a pressure differential across the valve (for example created by air, water or by an external mechanical element such as a pin). That is, the cleaning device can create a sufficient pressure differential across the valve to allow a fluid to enter and, as described below, to hydrate the powder with a liquid (e.g., water) and/or dispense the resultant slurry. The valves can be configured to remain permanently open once actuated, or they can be configured to close once the pressure differential is removed. In certain examples, the first port 494 and/or the second port 495 can comprise, for example, a plug valve, butterfly valve or a needle valve, or a resilient flap, pinch valve, cross slit valve, dome valve, etc. Once the valves have been actuated, the device can prevent removal of the cartridge until the cartridge contents have been consumed by the device.

In alternative embodiments, the first port 494 and/or the second port 495 can be a septum in the form of a frangible or puncturable membrane which can be pierced by a piercing component such as a needle or cannula and self-seals around the piercing component while it remains in place, and then self-seals substantially or completely when the piercing component is withdrawn. Typical materials for such frangible or puncturable membranes include butyl rubber or silicone rubber but any suitable material can be selected, having regards to its relative inertness to the intended contents of the cartridge. For ease of description, embodiments are primarily described herein with reference to first port 494 and/or the second port 495 as pressure actuated valves.

As shown in FIG. 4, the closure assembly further comprises a riser tube (e.g., conduit) 496 that extends from the first port 494 into the interior 405 of the tank 491. The riser tube 496 has a distal end 497 that, in this example, is adjacent the second end 403 of the tank 491. The riser tube 496 functions so that any flow through first port 494 takes place only through riser tube 496.

In the example of FIG. 4, the closure assembly 492 is configured to close the tank 491, and operates an interface with a lumen cleaning device 370 (e.g., a medical device lumen cleaning device, a food or beverage system cleaning device, a dental line cleaning device, etc.), and to support the internal structures in the cartridge which facilitate the mixing and dispensing of the slurry.

In certain embodiments, the cleaning agent cartridge 490 can be provided to the user containing water insoluble or partly water-soluble solid particles, optionally in a predetermined amount. The particles can be in a dry form and can be unaggregated, i.e., in particulate form. The solid particles can be present as a flowable solid and cleaning agent cartridge 490 can be a single-use consumable item.

As used herein, a water-soluble particle is one which has partial solubility in water, such that the particles in a saturated solution of the water soluble solid can be formed in a desired volume of water. One example of a suitable particulate material is sodium bicarbonate, Sodium bicarbonate can provide a useful solid volume fraction of a slurry with little wastage in the form of dissolved material. However, any other particulate material with suitable solubility can be used in accordance with embodiments of the invention, for instance, anhydrous sodium carbonate, which has a solubility of 30 g/L at 20° C., potassium carbonate, potassium bicarbonate, can also be used. Other compounds, including amino acids such as glycine can also be used.

Protection of the contents of the cleaning agent cartridge 490 from moisture prior to use can be a significant challenge. For example, in an example in which the solid material is sodium bicarbonate, even relatively low moisture contents can result in degradation of the contents. A low moisture permeability (e.g., less than 10mg/day/liter) may be desirable to prevent premature decomposition of sodium bicarbonate. In other cases, over time moisture can lead to caking or clumping of the contents which can adversely impact upon the flowability of the material and the ready mixing with the liquid component.

As such, in accordance with certain embodiments presented herein, is can be advantageous to provide a substantially airtight seal between the closure assembly 492 and the tank 491 to prevent moisture ingress at this junction. This airtight seal between the closure assembly 492 and the tank 491 can be provided in a number of different manners, such as via a screw-lock connection, press-fit, welding, etc. In addition, the cartridge components (e.g., the closure assembly 492 and the tank 491) can be formed from any one or more suitable moisture impermeable material, which is chemically inert to the initial contents and/or the slurry, and that has sufficient mechanical strength not only as packaging, but also for use with the cleaning device.

More specifically, during its life-cycle of the cleaning agent cartridge 490 can be conceptually understood to have two discrete states. In an initial state, the cleaning agent cartridge 490 operates as both a moisture barrier, and as physical protection to the contents for delivery to, and storage by, a user. However, as described further below, the cleaning agent cartridge 490 is also configured to engage/interface with a cleaning device. Once the cleaning agent cartridge 490 is engaged with the cleaning device, the cleaning agent cartridge 490 operates as a part of the fluidic system wherein the powder within the cleaning agent cartridge 490 can be hydrated with a liquid, such as water, and pressurized to aid with the delivery of a resulting slurry to a lumen. As such, the closure assembly 492 and the tank 491 are formed from materials that allow the cleaning agent cartridge 490 operate in both of these states (e.g., as packaging and as a component of a larger fluidic system).

In certain embodiments, the closure assembly 492 and/or the tank 491 can be formed from one or more polyethylene, polypropylene, polyethylene terephthalate, polystyrene, aluminum, glass, or aluminum or glass coated with polyethylene, polypropylene, polyethylene terephthalate, polytetrafluoroethylene, etc. In one specific example, high density polyethylene (HDPE) can be relatively effective as a moisture barrier in keeping out moisture to a level of less than 10 mg/day/liter.

Additionally, exposure to light and/or heat (above 60° Celsius) can result in decomposition of the contents. Controls can be put in place for temperature-controlled manufacture, storage and shipping of the product, as well as protection from light, by using light barrier material for the cartridge or a coating or wrapping of the cartridge.

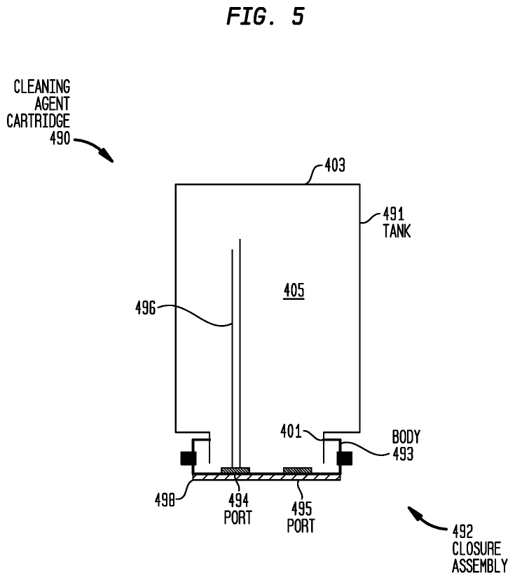

To prevent inadvertent exposure of the contents to moisture prior to the point of use, closure assembly 492 can be provided with an additional covering 498 (shown in FIG. 5) that can be removed by a user to expose the ports 494 and 495 immediately prior to engaging the cleaning agent cartridge 490 with the cleaning device. For example, the additional covering 498 can in the form of a liner (e.g., plastic or foil liner, a composition of several polymer layers with an aluminum foil layer, etc.) sealed around the circumference of the closure that the user can peel off. Transfer of dry powder can be another problem, particularly when done so in a high moisture environment. To address this problem, the cartridge operates as a powder reservoir when engaged to the device, thereby circumventing the issue of dry powder transfer. The device hydrates the dry powder (e.g., with a liquid such as water) creating the slurry, which is then conveyed from the cartridge into the device as required.

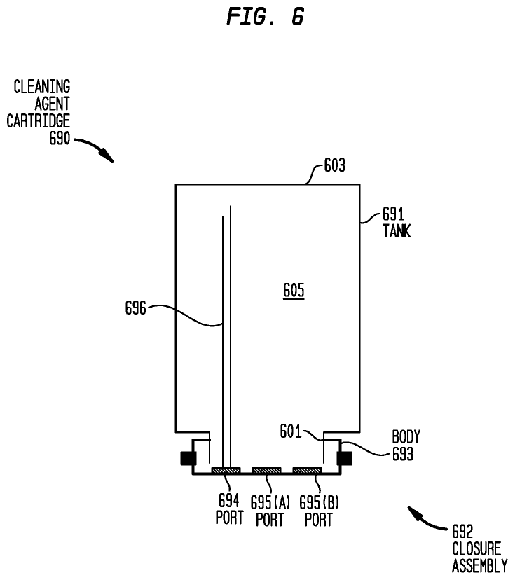

As noted, FIG. 4 illustrates an embodiment of a cleaning agent cartridge 490 comprising a first port 494 and a second port 495. Also as noted above, the presence of two ports is merely illustrative and that certain embodiments presented herein can include one or more additional first ports (e.g., each with an associated riser tube) and one or more additional second ports. For example, FIG. 6 is a schematic diagram illustrating a cleaning agent cartridge 690 comprising a tank portion (tank) 691 and a closure assembly 692. Similar to the embodiment of FIG. 4, the tank 691 comprises a first end 601, a second end 603, and defines an interior/internal volume 605. The closure assembly 692 comprises a body 693 having three (3) ports therein, referred to as a first port 694, second port 695A, and second port 695B. Also similar to FIG. 4, the body 693 is configured to be attached to the first end 601 of the tank 691, and the ports 694, 695A, and 695B are configured so as to close the internal volume 605 of the cartridge 690 (i.e., the region inside the tank 691) from uncontrolled contact with the atmosphere, water or other contaminants, while being operable to permit material to move in and/or out of the cartridge when required by a user or, for example, at a predetermined time in an automated cleaning cycle. The ports 694, 695A, and 695B can have any of the arrangements described above with reference to FIG. 4 and can be located at any position on the closure assembly 692.

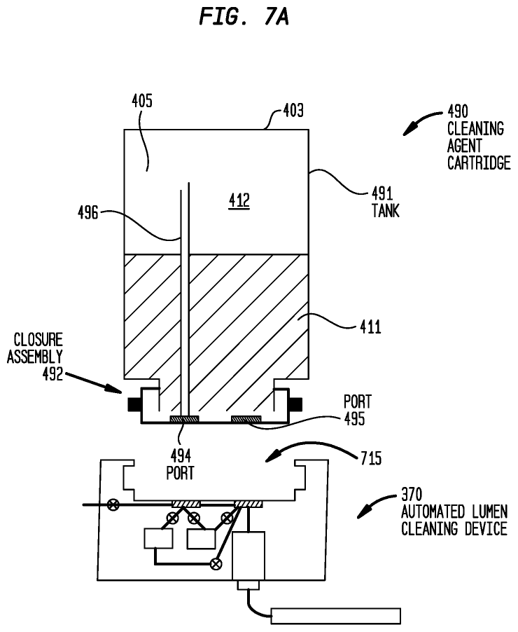

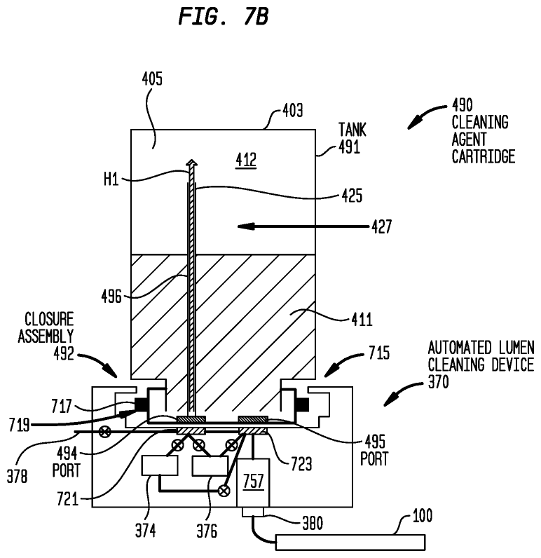

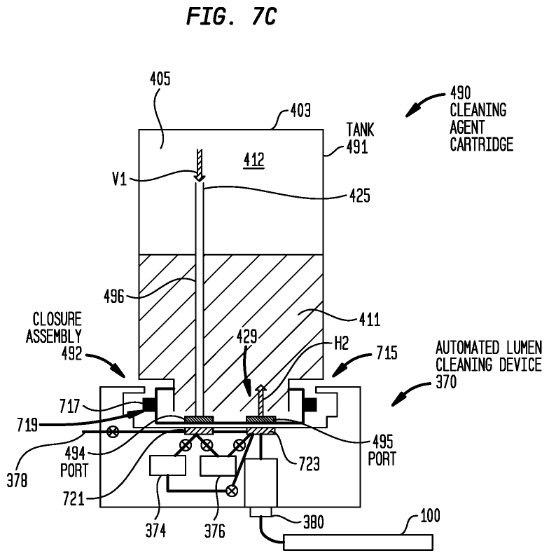

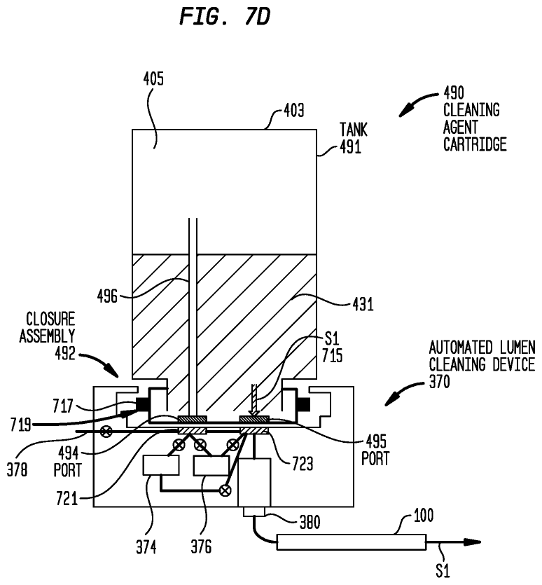

FIGS. 7A, 7B, 7C, and 7D are a series of schematic diagrams illustrating operation of a cleaning agent cartridge with a lumen cleaning device for mixing and dispensing a hydrated solid powder (slurry), in accordance with certain embodiments presented herein. Merely for ease of illustration, FIGS. 7A-7D will be described together and with reference to the cleaning agent cartridge 490 comprising first port 494 and second port 495 (FIG. 4) and lumen cleaning device 370 (FIG. 3). FIG. 7A illustrates the cleaning agent cartridge 490 separate from the lumen cleaning device 370, while FIGS. 7B-7D illustrate the cleaning agent cartridge 490 fluidically coupled to the lumen cleaning device 370. A device for cleaning, such as an endoscope 100, can be attached to the lumen cleaning device 370 contemporaneously with the cleaning agent cartridge 490.

In accordance with certain embodiments presented herein, the cleaning agent cartridge 490 initially has an amount of powder 411 dispensed into the tank 491 and the closure assembly 491 is then attached to the tank 491 to close the tank. The cleaning agent cartridge 490 can be stored, transported, and/or used in any orientation. However, in certain examples, the cleaning agent cartridge 490 will typically be positioned in a lumen cleaning device, such as lumen cleaning device 370, in such a manner that the closure assembly 491 is retained as a base portion that is disposed inferior to the tank 491 (e.g., in use, the tank 491 is disposed above the closure assembly 491). This orientation can be advantageous for venting of the cartridge 490 during the mixing, if required, and dispensing of the hydrated powder (slurry).

As noted, FIG. 7A shows that the dry powder 411 is retained in the tank 491, and that the riser tube 496 extends above the dry powder when the cartridge 490 is in an orientation in which the closure assembly 492 is inferior to the tank 491. However, the tank 491 also includes a volume 412 of gas (e.g., air) to permit free movement of the dry powder 411. In an alternative embodiment (not shown), the powder 411 could extend above the riser tube 496 when the cleaning agent cartridge 490 is in an orientation in which the closure assembly 492 is inferior to the tank 491.

As shown in FIGS. 7B and 7C, the cleaning agent cartridge 490 is fluidically coupled to the lumen cleaning device 370, where the cleaning agent cartridge 490 can be, for example, inside of, on a surface of, etc. of the lumen cleaning device 370. In this example, the lumen cleaning device 370 includes a socket 715 to receive cleaning agent cartridge 490 (e.g., the closure assembly 492) and to retain it in a defined position such that the ports 494 and 495 in the closure assembly 492 are located such that they can be accessed by port interfaces 721 and 723, respectively, of the lumen cleaning device 370. The port interfaces 721 and 723 can comprise openings/apertures configured to fluidically mate/couple with the respective ports 494 and 495 in the closure assembly 492 (e.g., provide a substantially fluid-tight seal with the ports 494 and 495). Additionally, in certain embodiments, the port interfaces 721 and 723 can include valve actuators that operate to open the ports 494 and 495.

In certain embodiments, the port interfaces 721 and/or 723 can receive a supply of a gas or liquid at a sufficient pressure to actuate/open the ports 494 and 495. For example, the gas or liquid can be supplied to create a pressure differential applied to the valves by the gas or liquid from the cleaning device serves to open the ports 494 and 495 and permit direct flow of gas or liquid into the cartridge 490, or serve to propel gas, liquid or the slurry from the cartridge 490. Alternatively, the port interfaces 721 and/or 723 can comprise a mechanical component for example a push rod, flap, cylinder or other known mean to thus enable fluid communication between the lumen cleaning device 370 and the tank 491 and to control the flow of material into and out of the cartridge 490.

As noted above, it is to be appreciated that a cleaning agent cartridge in accordance with embodiments presented herein can include multiple first and/or second ports (for instance, one first port and two second ports) and these can be actuated by respective multiple correspondingly located valves on the device. In some cases where multiple ports are present, one or more ports can remain unactuated or blind. This arrangement enables a single cartridge configuration to be used in differently configured cleaning devices.

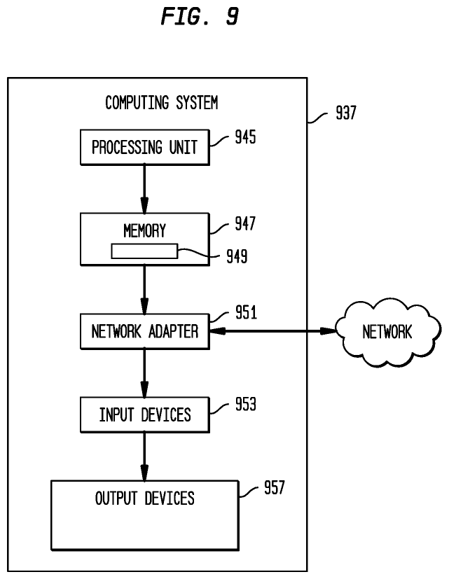

In the illustrated embodiment of FIGS. 7A-7C, the lumen cleaning device 370 is configured to dispense, in a controlled manner, gas (e.g., air) from a gas source via the gas connector 374 and water from water source via water connector 376 into the cartridge 490 (e.g., via port interface 721 and first port 494). The lumen cleaning device 370 is also configured to vent and/or drain fluids from via drain lines and the device drain fitting 378. In addition, the lumen cleaning device 370 is configured to control various mixing parameters, such as the air and water pressure and the time these are applied to the cartridge 490, the total volume and flow rate of air and water into and out of the cartridge 490, etc. In certain examples, the lumen cleaning device 370 operates under control of a computing device. Also, the lumen cleaning device 370 is able to control the egress of the slurry from the cartridge 490 via second port 495 and the port interface 723 and direct and/or propel the fluidic cleaning composition into a lumen of the endoscope 100 (via connector 380), again at a controlled flow rate which is most usually under control of a computing system (e.g., as shown in FIG. 9). In certain examples, the slurry is propelled into a lumen of the endoscope 100 by a delivery mechanism 757. In certain examples, the delivery mechanism can be implemented as described in commonly owned International Patent Application No. PCT/AU2022/050568, filed Jun. 9, 2022, the content of which is hereby incorporated by reference herein.

As noted above, the cleaning agent cartridge 490 engages with the lumen cleaning device 370, for example by way of a socket 715 which is configured (e.g., shaped and/or sized) to mate with at least a part of an external portion of the closure assembly 492. The closure assembly 492 and/or the socket 715 can further comprise location, engagement and/or retention components to assist in positioning the cartridge 490 in the correct position. For example, the closure assembly 492 can contain locating one or more lugs 717 which facilitate a bayonet type connection with the device through complementary slots 719.

It is to be appreciated that the specific lug and slot engagement is merely illustrative and that a cleaning agent cartridge could engage with a lumen cleaning device in other manners. For example, in an alternative embodiment, the closure assembly 492 could include external threads configured to mate with correspond threads of the socket 715 with a stop to ensure the correct orientation of the ports with the valves in the lumen cleaning device 370. In certain examples, the lumen cleaning device 370 can include a locking mechanism (not shown) to prevent manual removal of the cleaning agent cartridge 490 from the device until the mixing, dispensing, and where used, cleaning, cycles are completed.

Once the cleaning agent cartridge 490 is fluidically coupled to the lumen cleaning device 370, the ports 494 and/or 495 are actuated in a desired manner, by one or more of the port interfaces (e.g., valve actuators) 721 and/or 723, respectively, allowing the device to hydrate the contents of the tank 491. More specifically, in one example, port interface 721 actuates and opens first port 494, placing the aperture of the port interface 721 in fluid communication with the riser tube 496. The port interface 721 allows for the flow of gas or liquid into the cartridge 490.

FIGS. 7B, 7C, and 7D show the cartridge 490 in use and attached to the lumen cleaning device 370, but each of these drawings represent different stages/phases operation. Referring first to the phase represented in FIG. 7B, in this example the first port 494 has been opened by port interface 721 and water enters the tank 491 via the riser tube 496. As such, the hydration of the powder 411 commences at what is referred to as herein as a “first hydration locus” 427 that is adjacent the distal end 425 of the riser tube 496. That is, water is caused to flow via port interface 721 and the path of the hydration water is shown as H1 (i.e., initially via riser tube 496). The water then exits the hydration path at the distal end 425 of the riser tube and makes first contact with the dry powder 411 adjacent thereto. In this phase, the hydration path thus extends into the tank and the first hydration locus is located a distance from the ports 494 and 495. For example, the first hydration locus can be located at a mid-point of the tank 491, or further away from the ports 494 and 495, e.g., 50%, 75% or more of the distance towards the distal end of the tank relative to the closure assembly 492. Stated differently, the first hydration locus can be located within a distal ½, a distal ⅓, or a distal ¼ of the tank 491. The flow of water into first port 494 is then ceased after, for example, a period of time, delivery of an amount of water, etc.

Referring next to FIG. 7C, once hydration water has been provided by first port 494, that flow ceases and second port 495 is opened by port interface 723 and the hydration of the powder 411 can subsequently commence at what is referred to as a “second hydration locus” 429. In this example, water is caused to flow via port interface 723 and, as shown, the water enters near the proximal end 401 of the tank. The path of the hydration water in this case is shown as H2. Contemporaneously, first port 494 can re-open (e.g., via pressure build-up), thereby allowing the venting of gas at the distal end 403 of the cartridge 490 via riser tube 496. The venting flow, which can exit via device drain fitting 378, is generally shown by arrow V1. The flow of water into second port 495, and the flow of air out of first port 494, is then ceased after, for example, a period of time, delivery of an amount of water, etc.

As noted, FIGS. 7B and 7C illustrate first and second hydration phases, respectively, that hydrate the powder 411 from two discrete locations (i.e., the first hydration locus 427 and second hydration locus 429). It is to be appreciated that the first and second hydration phases represented by FIGS. 7B and 7C can be repeated, for example, in an alternating sequence, a number of times until the initial powder 411 is sufficiently hydrated to form the slurry. The number of times that each of the first and second hydration phases are performed can vary, for example, with different size containers, different powders, different hydration parameters, etc.

In certain examples, a control loop can operate to set the repetition/iteration of the first and second hydration phases. For example, a control system can control operation based on predetermined data or, in certain cases, using real-time data measurements, such as water flow rate, pressure data (e.g., from pressure sensors in the container and/or the cleaning device), rheology data (e.g., data relating to the flow of the output from the cartridge), etc. The control can set, for example, the number of repeats of the first and second hydration phases, time periods over which the water is introduced into the cartridge (e.g., open first port 494 for 2 seconds), etc.

It is also to be appreciated that the first and second hydration phases, as represented by FIGS. 7A and 7B, may not be performed sequentially and, instead, could be performed substantially simultaneously in some embodiments. For example, in an alternative embodiment, first port 494 and second port 495 can be simultaneously opened and the hydration of the powder 411 can commence at both the first hydration locus and second hydration locus at the same time.

As noted, FIGS. 7B and 7C illustrate that the cartridge 490 is configured for hydration of the solid powder 411 contained therein at two separated hydration loci, via different pathways H1 and H2. This serves to address the fact that single point hydration from the outside of the solid bolus can present problems. For example, dispensing water from the top alone can lead to the formation of trapped air pockets, while dispensing from the bottom alone can lead to floating pockets of solid, which can both ultimately result in inefficient and/or partially-homogenized hydration of the contents The introduction of water at two loci can reduce the potential for clumping and further assists in the mixing of the water with the solid powder 411. Even if poor solid liquid mixing is relatively temporary, it can skew the relative water/solid ratios exiting the cartridge. Good mixing of the solid and liquid can thus be important, and it should be borne in mind that while mixing can be checked and assisted in manual processes, in automated processes, it can be beneficial for the mixing to give reproducible and predictable outcomes every time. In certain examples, the amount of water can be calculated taking into account the amount of powder present in the tank and the target solid fraction required for the slurry.

Returning to the embodiments of FIGS. 7A-7D, once the hydration of powder 411 has reached a desired level by way of the addition of a suitable amount of water, the addition of water is terminated and the resultant slurry can be dispensed for use in cleaning, for example, a lumen of the endoscope 100. FIG. 7D illustrates the contents of the cleaning agent cartridge 490 as the resultant slurry 431 (i.e., the hydrated powder). As noted elsewhere herein, in certain examples of the slurry 431, the water has partially dissolved the solid powder 411 to form a saturated solution into which no further solid would dissolve, and the solid particles are suspended. The slurry can experience some settlement and there can be a small layer of water or saturated solution above the slurry but this can be taken into account and the relative effect can be adjusted beforehand.

In certain embodiments, as shown in FIG. 7D, the slurry is dispensed via second port 495 for use by the cleaning device in cleaning the endoscope 100. In certain embodiments, port interface 721 can be used to introduce air and/or water from via first port 494. As the air and/or water enters the cartridge, flowing hydration path H1, there may be some compression of air in the headspace, but in general, the water introduced via first port 94 displaces slurry 431 via port 495. The orientation of the exit port 495 at the inferior end of the container 490 ensures that slurry 431 is dispensed to the cleaning device 370. The slurry 431 exits via path S1 and can be propelled by the delivery mechanism 757 into endoscope 100, where it carries out the desired cleaning, and then exits to waste.

In certain embodiments, the flow of the slurry 431 can be regulated by the cleaning device 370, namely delivery mechanism 757. For example, the delivery mechanism 757 could be provided with (e.g., via a pump) an apportioned amount of the slurry 431 from the cartridge 490 and then propel the apportioned amount of the slurry 431 into the lumen of the endoscope 100. Commonly owned International Patent Application No. PCT/AU2022/050568, filed Jun. 9, 2022, the content of which is hereby incorporated by reference herein, describes example delivery mechanisms that can used with cartridge 490. In alternative embodiments, the slurry 431 can be propelled into a lumen of the endoscope 100, for example, directly by the pressure generated by the air and/or water entering the container 490 via first port 494.

As described elsewhere herein, the slurry 431 can be dispensed in a number of different manners. For example, the slurry 431 can be dispensed in apportioned amounts selected/configured to a specific lumen to be cleaned. As a result, the slurry 431 can be dispensed over a number of cycles.

In certain embodiments, once the slurry 431 has been dispensed from the cartridge, the cartridge can be rinsed with water, drained and dried via one or more of the ports. That is, the cartridge 490 can be rinsed with water (e.g., via port 494), drained (e.g., via port 495), and blown dry by air (e.g., via port 494 and/or 495). In this way, the cartridge can then be removed substantially clean and dry, which can improve convenience and safety.

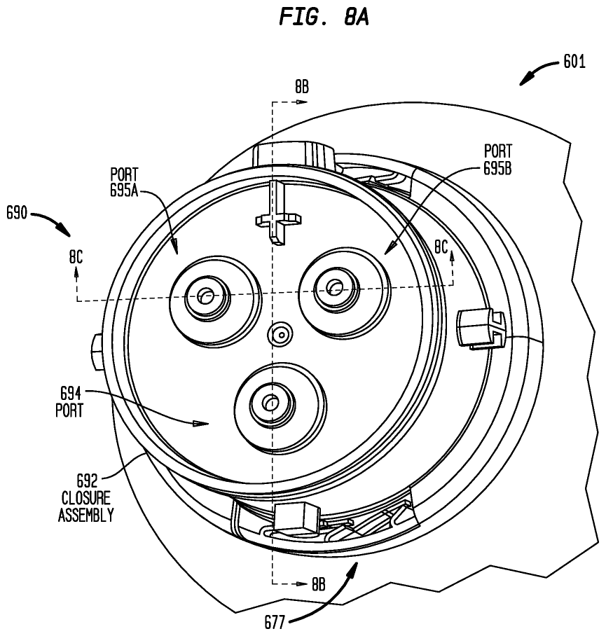

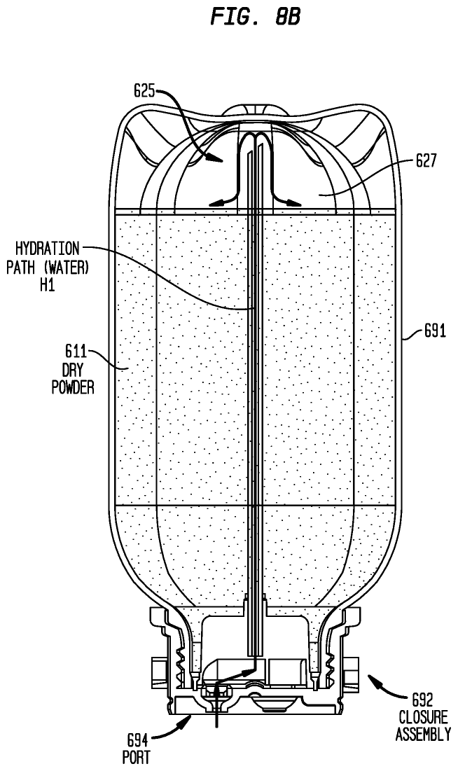

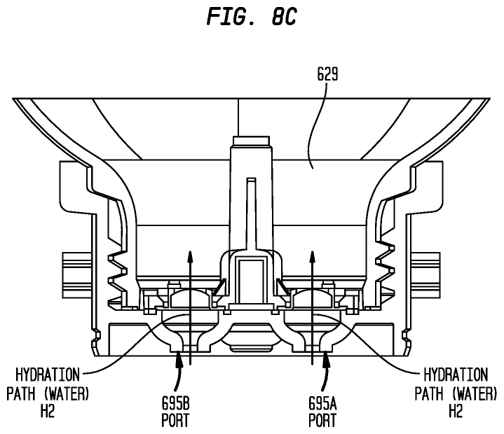

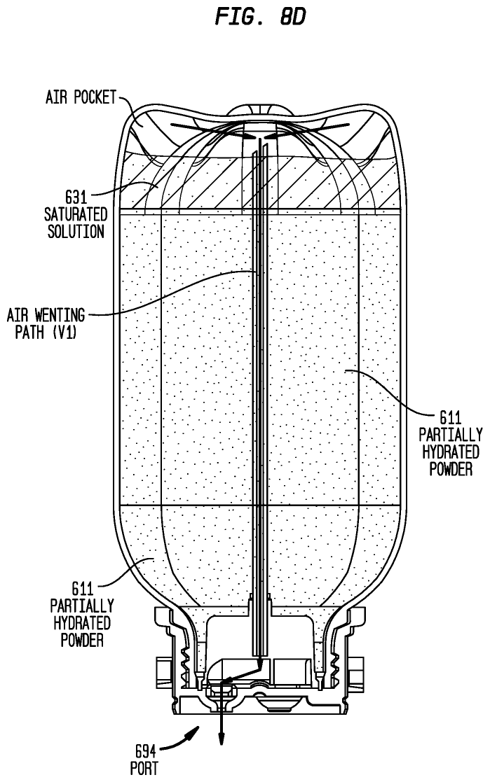

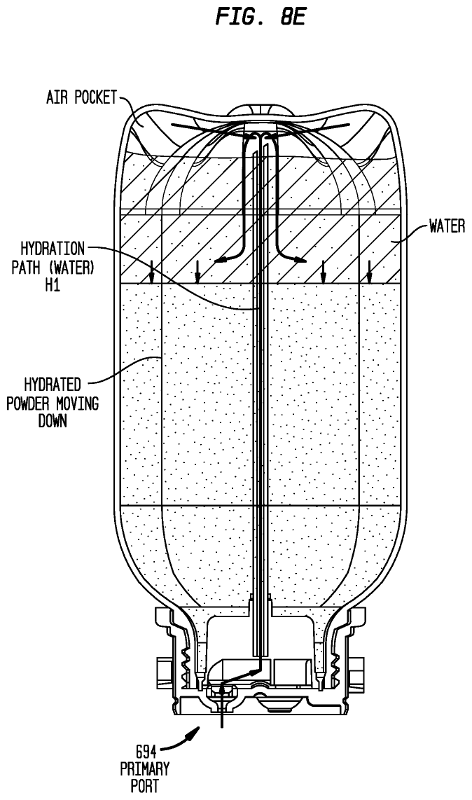

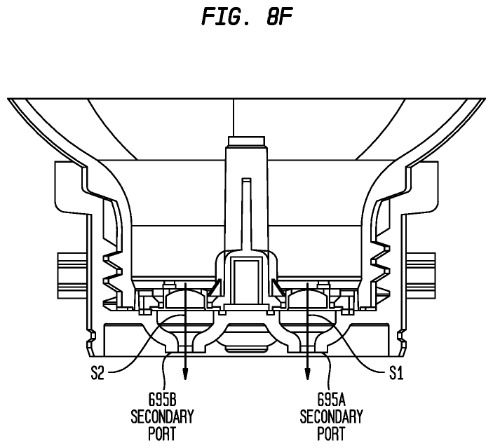

As noted above, FIGS. 7A-7D have been described with reference to an embodiment in which the cartridge 490 includes a single first port and a single second port. However, also as noted above, this specific arrangement is merely illustrative and that, in alternative embodiments, multiple first ports and/or multiple second ports can be present. FIG. 6 illustrates one such embodiment in which the cartridge 690 comprises one first port 694 and two second ports, referred to as second ports 695A and 695B. FIGS. 8A-8F are a series of diagrams illustrating operation of the cartridge 690. For ease of illustration, FIGS. 8A-8F will be described together and the cartridge 690 is shown separate from an associated cleaning device.

Referring first to FIG. 8A, shown is a perspective via of a portion of the cartridge 690, namely the closure assembly 692 and the first (proximal) end 601 of the tank 691. Shown in FIG. 8A is the first port 694, the second port 695A, and the second port 695B. Also shown in FIG. 8A are anti-tamper features 677 on the closure assembly 692 that prevent the closure assembly 692 from being disconnected (e.g., unscrewed from) the tank 691, which in turn helps protect the contents of the cartridge from moisture ingress. In one form, the tamper features 677 are in form of teeth that are configured that are engageable with a complementary set of teeth provided on the tank 691.

In certain embodiments, the first port 694, the second port 695A, and the second port 695B are formed from thermoplastic elastomer materials, and plastic fastening features (e.g., plastic anti-tamper features 677) are used with the closure assembly 692. The use of these materials may enable the entire cartridge 690 (e.g., tank 691 and closure assembly 692) to be recycled without disassembly/segregation of the individual components

FIG. 8B is a cross-sectional view of the cartridge 690 taken along section line 8B-8B of FIG. 8A, and represents a first phase/stage of a hydration process. In this example, water is added via first port 694, while second ports 695A, 695B remain closed. More specifically, in this example the first port 694 has been opened and water enters the tank 691 via the riser tube 696. As such, the hydration of the powder 611 commences at a “first hydration locus” 627 that is adjacent the distal end 625 of the riser tube 696. That is, the water exits the hydration path at the distal end 625 of the riser tube 696 and makes first contact with the dry powder 611 adjacent thereto. In this phase, the hydration path thus extends into the tank and the first hydration locus is located a distance from the ports 694, 695A, and 695B. For example, the first hydration locus can be located at a mid-point of the tank 691, or further away from the ports 694, 695A, and 695B, e.g., 50%, 75% or more of the distance towards the distal end of the tank relative to the closure assembly 692. Stated differently, the first hydration locus 627 can be located within a distal ½, a distal ⅓, or a distal ¼ of the tank 691. The flow of water into first port 694 is then ceased after, for example, a period of time, delivery of an amount of water, when a pressure set point has been met, etc.

FIG. 8C is a cross-sectional view of the cartridge 690 taken along section line 8C-8C of FIG. 8A representing one aspect of a second phase/stage of the hydration process, while FIGS. 8B, 8D, and 8E are cross-sectional views of the cartridge 690 taken along section line 8B-8B of FIG. 8A representing another aspect of the second phase/stage of the hydration process. In this second stage, air is purged from the distal end of the cartridge 690 via first port 694 (open to atmosphere via a drain line), while water is added via second ports 695A and 695B.

More specifically, as shown in FIG. 8C, once hydration water has been provided by first port 694 in the first stage, that flow ceases and second ports 695A and 695B are opened and the hydration of the powder 611 can subsequently commence at what is referred to as a “second hydration locus” 629. In this example, the water enters near the proximal end 601 of the tank and the two paths of the hydration water in this case are shown as H2. Contemporaneously, as shown in FIG. 8D, first port 694 can re-open, thereby allowing the venting of fluids (e.g., gas or the saturated solution) from the distal end 603 of the cartridge 690 via riser tube 696. The venting flow is generally shown by arrow V1. The flow of water into second ports 695A and 695B, and the flow of air out of port 694, is then ceased after, for example, a period of time, delivery of an amount of water, etc.

As noted, FIG. 8B illustrates a first hydration stage associated with cartridge 690 (e.g., hydration at a first hydration locus 427), while FIGS. 8C and 8D collectively illustrate a second hydration stage associated with cartridge 690 (e.g., hydration a second hydration locus 429). Notably, FIG. 8D illustrates a state of contents of the cartridge 690 has having two simultaneous parts, including a first part comprising partially hydrated powder 611 and a second part comprising saturated solution forming a fluidic cleaning composition 631. This specific state of contents of the cartridge 690 can represent, for example, the contents after one or two iterations of the first hydration stage and the second hydration stage. In practice, the first and second hydration phases can be repeated, for example, in an alternating sequence, a number of times until the initial dry powder 611 is sufficiently hydrated to form the slurry 631. The number of times that each of the first and second hydration phases are performed can vary, for example, with different size containers, different powders, different hydration parameters, etc.

Once the hydration of powder 611 has reached a desired level by way of the addition of a suitable amount of water, the addition of water is terminated and the resultant fluidic slurry 631 can be dispensed for use in cleaning, for example, a lumen of an endoscope. FIGS. 8E and 8F illustrate the contents of the cleaning agent cartridge 690 as the resultant slurry 631 (i.e., the hydrated powder), and dosing/dispensing of the slurry 631. In this example, water is delivered via first port 694 and the slurry 631 is dispensed via second port 695A and/or second port 695B.

In certain embodiments, the slurry 631 can be propelled into a lumen, for example, by a delivery mechanism within the lumen cleaning device. For example, the delivery mechanism could extract (e.g., pump) an apportioned amount of the slurry 631 from the cartridge 690 and then propel the apportioned amount of the slurry 631 into the lumen of the endoscope 100 via port 695A and/or port 695B, shown as exit path S1 and S2. Commonly owned International Patent Application No. PCT/AU2022/050568, filed Jun. 9, 2022, the content of which is hereby incorporated by reference herein, describes example delivery mechanisms that can used with container 690 to propel an apportioned amount of the slurry 631 into a lumen.

In alternative embodiments, the slurry 631 can be propelled into a lumen of the endoscope 100, for example, by pressure generated by the air and/or water entering the container 790 via first port 694. In one such embodiment, a fluid enters the cartridge 690 following hydration path H1 via first port 694 and displaces an amount of slurry 631. The displaced slurry 631 is dispensed via port 695A and/or port 695B, shown as exit path S1 and S2. The orientation of the exit port(s) 695A and/or 695B at the inferior end of the container 690 ensures that slurry 631 is dispensed to the cleaning device.

In general, the embodiment of FIGS. 8A-8F, which includes a plurality of second ports 695A and 695B, can allow for faster egress of slurry from the container 690. The multiple second ports 695A and 695B can be used to provide multiple slurry flows for cleaning multiple endoscope channels and/or multiple endoscopes, or multiple slurry flows can be recombined to provide a higher flow volume of the slurry 631.

As noted, for ease of description, aspects of the techniques presented herein have generally been described above with reference to a cleaning agent in the form of a powder, and the dispensing of a fluidic cleaning composition in the form of a slurry. However, it is to be appreciated that the techniques presented herein can be used with other types of cleaning agents and other types of fluidic cleaning compositions. For example, in one alternative embodiment, the initial contents of the container could be a concentrated liquid (e.g., disinfectant) to which a fluid (e.g., water) is added dilute the concentrated liquid to an appropriate levels for use as a cleaning composition. In other words, in the above description, the powder could, potentially with some modifications to the workflow, be substituted for a liquid or other cleaning agent to form a resultant fluidic cleaning composition for dispensing and use in cleaning, for example, a lumen.

As described elsewhere herein, the hydration of a clearing agent (e.g., powder) in a cleaning agent cartridge, as well as the dispensing of a resultant fluidic cleaning composition, can be controlled by a control system. FIG. 9 is block diagram illustrating an example computing device 917 configured to operate as a control system for hydration and dispensing, as described elsewhere herein. The computing device 937 also operates as a control system for a lumen cleaning device. The computing device 937 can comprise, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 937 can be a single virtual or physical device operating in a networked environment over communication links to one or more remote devices, such as an implantable medical device or implantable medical device system.

In its most basic configuration, computing device 937 includes at least one processing unit 945 and memory 947. The processing unit 925 includes one or more hardware or software processors (e.g., Central Processing Units) that can obtain and execute instructions. The processing unit 945 can communicate with and control the performance of other components of the computing system 937.

The memory 937 is one or more software or hardware-based computer-readable storage media operable to store information accessible by the processing unit 945. The memory 947 can store, among other things, instructions executable by the processing unit 945 to implement applications or cause performance of operations described herein, as well as other data. The memory 947 can be volatile memory (e.g., RAM), non-volatile memory (e.g., ROM), or combinations thereof. The memory 947 can include transitory memory or non-transitory memory and/or one or more removable or non-removable storage devices. In examples, the memory 947 can include RAM, ROM, EEPROM (Electronically-Erasable Programmable Read-Only Memory), flash memory, optical disc storage, magnetic storage, solid state storage, or any other memory media usable to store information for later access. In examples, the memory 947 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 947 can include wired media such as a wired network or direct-wired connection, and wireless media such as acoustic, RF, infrared and other wireless media or combinations thereof. In certain embodiments, the memory 947 comprises cartridge control logic 949 that, when executed, enables the processing unit 945 to perform aspects of the techniques presented.

In the illustrated example, the system 937 further includes a network adapter 951, one or more input devices 953, and one or more output devices 955. The system 937 can include other components, such as a system bus, component interfaces, a graphics system, a power source (e.g., a battery), among other components. The network adapter 951 is a component of the computing system 937 that provides network access (e.g., access to at least one network). The network adapter 951 can provide wired or wireless network access and can support one or more of a variety of communication technologies and protocols, such as ETHERNET, cellular, BLUETOOTH, near-field communication, and RF (Radiofrequency), among others. The network adapter 931 can include one or more antennas and associated components configured for wireless communication according to one or more wireless communication technologies and protocols.

The one or more input devices 953 are devices over which the computing system 937 receives input from a user. The one or more input devices 953 can include physically-actuatable user-interface elements (e.g., buttons, switches, or dials), touch screens, keyboards, mice, pens, and voice input devices, among others input devices. The one or more output devices 955 are devices by which the computing system 937 is able to provide output to a user. The output devices 955 can include, a display, one or more speakers, among other output devices.

It is to be appreciated that the arrangement for computing system 937 shown in FIG. 9 is merely illustrative and that aspects of the techniques presented herein can be implemented at a number of different types of systems/devices. For example, the computing system 937 could be a laptop computer, tablet computer, mobile phone, surgical system, etc.

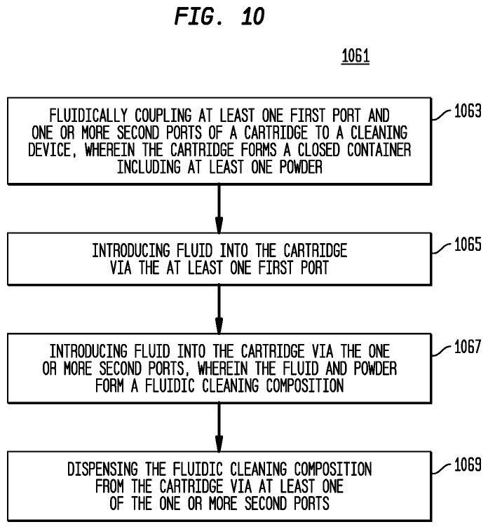

FIG. 10 is flowchart illustrating an example method 1061, in accordance with certain embodiments presented herein. Method 1061 begins at 1063 where at least one first port and one or more second ports of a cartridge, which forms a closed container including at least one powder, are fluidically coupled to a cleaning device. At 1065, the cleaning device introduces fluid into the cartridge via the at least one first port to at least partially hydrate the at least one powder. At 1067, the cleaning device introduces fluid into the cartridge via the one or more second ports to at least partially hydrate the at least one powder. The fluid and powder form a fluidic cleaning composition. At 1069, the fluidic cleaning composition is dispensed from the cartridge via at least one of the one or more second ports.

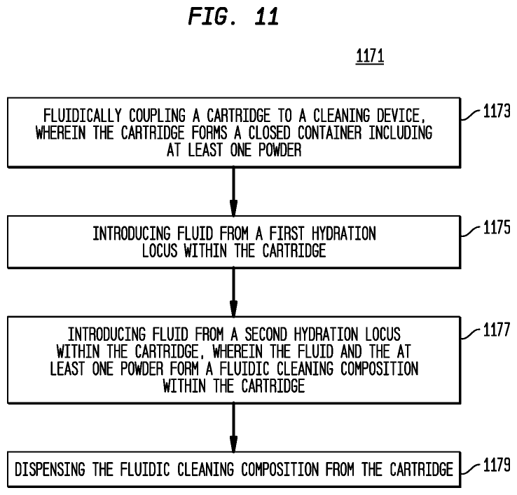

FIG. 11 is flowchart illustrating another example method 1161, in accordance with certain embodiments presented herein. Method 1161 begins at 1163 where a cartridge, which forms a closed container and includes at least one powder, is fluidically coupled to a cleaning device. At 1165, fluid is introduced from a first hydration locus within the cartridge. At 1167, fluid is introduced from a second hydration locus within the cartridge. The fluid and at least one powder form a fluidic cleaning composition. At 1169, the fluidic cleaning composition is dispensed from the cartridge.

The cleaning agent cartridge and associated techniques presented herein can provide a number of advantages over other cartridge systems. For example, in accordance with certain aspects presented herein, the hydration of the solid powder occurs at two locations, including a location adjacent a proximal end of the container and a more distal location (e.g., a center or distal portion of the container). The use of two hydration loci enables the fluid (e.g., water) to contact the bolus of solid material at two points, thereby facilitating even mixing. In addition, attachment of the cartridge via only a closure assembly can be beneficial. In the present case, the volume required to clean a lumen (e.g., endoscope lumen) is not insignificant so a mechanism with a fully internal cartridge would require a significant increase in form factor. A single point of attachment of the cartridge also facilitates alignment and removal and minimizes the potential for breakage of the cartridge or cleaning device.

Moreover, in accordance with embodiments presented herein, the mixing of the powder with the fluid takes place entirely within the cartridge, the outside of which remains dry. A number of conventional cartridge mixing, and extraction systems result in the cartridge being wet after use and during removal. Inconvenience and mess aside, in an environment where decontamination and reprocessing of medical instruments takes place, large quantities of water could lead to cross contamination on the capsule during handling if the user has ‘dirty’ hands or gloves. By virtue of being fluidically coupled, no dispensing/metering of dry powder is required. Finally, the present cartridge provides a plurality of ports operable to allow fluid ingress and egress, and which can thereby each provide a variety of functionality as discussed.

1-59. (canceled)

60. A method for use of a cartridge that defines a closed container including at least one powder, comprising:
introducing fluid from a first hydration locus within the cartridge;
introducing fluid from a second hydration locus within the cartridge,
wherein the fluid and the at least one powder form a fluidic cleaning composition within the cartridge; and
dispensing the fluidic cleaning composition from the cartridge.

61. The method of claim 60, further comprising:
iteratively introducing fluid into the cartridge from the first hydration locus and from the second hydration locus in an alternating sequence.

62. The method of claim 61, further comprising:
controlling a number of iterations of the alternating sequence based on one or more real-time measurements.

63. The method of claim 60, further comprising:
substantially simultaneously at introducing fluid into the cartridge from the first hydration locus and from the second hydration locus in an alternating sequence.

64. The method of claim 60, further comprising:
venting gas from the closed container while introducing fluid from the second hydration locus within the cartridge.

65. The method of claim 60, wherein the cartridge has a proximal end and a distal end, and wherein introducing fluid from a first hydration locus within the cartridge comprises:
introducing fluid from a site located relatively closer to the distal end than the proximal end.

66. The method of claim 60, wherein the cartridge has a proximal end and a distal end, at least one first port located at the proximal end of the cartridge, and a riser tube attached to the at least one first port, and wherein introducing fluid from a first hydration locus within the cartridge comprises:
introducing fluid via a distal end of the riser tube.

67. The method of claim 60, wherein the cartridge has a proximal end and a distal end, and wherein introducing fluid from a second hydration locus within the cartridge comprises:
introducing fluid from a site located relatively closer to the proximal end than the distal end.

68. The method of claim 60, wherein the cartridge has a proximal end and a distal end, and one or more second ports located at the proximal end of the cartridge, and wherein introducing fluid from a second hydration locus within the cartridge comprises:
introducing fluid via the one or more second ports.

69. The method of claim 60, wherein dispensing the fluidic cleaning composition from the cartridge from the cartridge comprises:
opening one or more second ports in the cartridge; and
releasing the fluidic cleaning composition via the one or more second ports.

70. The method of claim 69, wherein the one or more second ports comprise a plurality of ports, and wherein releasing the fluidic cleaning composition via the one or more second ports comprises:
releasing the fluidic cleaning composition via each of the plurality of ports.

71. The method of claim 70, wherein releasing the fluidic cleaning composition via each of the plurality of ports comprises:
sequentially releasing the fluidic cleaning composition via the plurality of ports.

72. The method of claim 70, wherein releasing the fluidic cleaning composition via each of the plurality of ports comprises:
contemporaneously releasing the fluidic cleaning composition via the plurality of ports.

73. The method of claim 60, wherein dispensing the fluidic cleaning composition from the cartridge comprises:
introducing fluid into the cartridge to expel a corresponding volume of the fluidic cleaning composition via one or more ports.

74. The method of claim 60, wherein dispensing the fluidic cleaning composition from the cartridge comprises:
pumping an amount of the fluidic cleaning composition out of the cartridge via one or more ports.

75. A method, comprising:
fluidically coupling at least one first port and one or more second ports of a cartridge to a cleaning device, wherein the cartridge forms a closed container including at least one cleaning agent;
introducing fluid into the cartridge via the at least one first port;
introducing fluid into the cartridge via the one or more second ports,
wherein the fluid and at least one cleaning agent form a fluidic cleaning composition; and
dispensing the fluidic cleaning composition from the cartridge via at least one of the one or more second ports.

76. The method of claim 75, further comprising:
iteratively introducing fluid into the cartridge via the at least one first port and via the one or more second ports in an alternating sequence.

77. The method of claim 76, further comprising:
controlling a number of iterations of the alternating sequence based on one or more real-time measurements.

78. The method of claim 75, further comprising:
contemporaneously introducing fluid into the cartridge via the at least one first port and via the one or more second ports.

79. The method of claim 75, further comprising:
venting fluid from the closed container while introducing fluid into the cartridge via the one or more second ports.
